(19) United States
(12) Patent Application Publication
(10) Pub. No.: US 2019/0082981 A1
(43) Pub. Date: Mar. 21, 2019

(54) FINGER CUFF TO APPLY CONCENTRATED PRESSURE TO THE BOTTOM OF A PATIENT'S FINGER

(71) Applicant: Edwards Lifesciences Corporation, Irvine, CA (US)

(72) Inventors: Peiyuan Li, Amsterdam (NL); Olaf Schraa, Amsterdam (NL); Geertruida Lucretia van der Sar, Alphen aan den Rijn (NL)

(21) Appl. No.: 16/129,619

(22) Filed: Sep. 12, 2018

Related U.S. Application Data
(60) Provisional application No. 62/560,415, filed on Sep. 19, 2017.

(51) Int. Cl. A61B 5/022; A61B 5/024

(52) U.S. Cl. CPC A61B 5/02241; A61B 5/0059; A61B 5/02427; A61B 5/02422

(57) ABSTRACT

Disclosed is a finger cuff attachable to a patient's finger to be used in measuring the patient's blood pressure by a blood pressure measurement system utilizing the volume clamp method. The finger cuff may include a light emitting diode (LED) and photodiode to form an LED-PD pair to perform measurements of a plethysmogram signal to aid in measuring the patient's blood pressure; and a bladder. The bladder includes a bottom portion that abuts only the bottom portion of the patient's finger to apply concentrated pressure only on the bottom of the patient's finger near both of the patient's two arteries, wherein, when the finger cuff is placed around the patient's finger, the bottom portion bladder and LED-PD pair aid in measuring the patient's blood pressure by the blood pressure measurement system.

### CROSS-REFERENCE TO RELATED APPLICATION

This application claims priority to U.S. Provisional Application No. 62/560,415, filed Sep. 19, 2017, the contents of which are incorporated herein by reference.

### BACKGROUND

### Field

Embodiments of the invention relate generally to non-invasive blood pressure measurement. More particularly, embodiments of the invention relate to a finger cuff to apply concentrated pressure to the bottom (e.g., ‘bottom’ side denotes here the palmar side) of a patient's finger.

### Relevant Background

Volume clamping is a technique for non-invasively measuring blood pressure in which an external pressure is applied to a patient's finger in such a manner that arterial pressure may be balanced by a time varying pressure to maintain a constant arterial volume. In a properly fitted and calibrated system, the applied time varying pressure is equal to the arterial blood pressure in the finger. The applied time varying pressure may be measured to provide a reading of the patient's arterial blood pressure.

This may be accomplished by a finger cuff that is arranged around a finger of a patient. The finger cuff may include an infrared light source, an infrared sensor, and an inflatable bladder. The infrared light may be sent through the finger in which a finger artery is present. The infrared sensor picks up the infrared light and the amount of infrared light registered by the sensor may be inversely proportional to the artery diameter and indicative of the pressure in the artery.

In the finger cuff implementation, by inflating the bladder of the finger cuff, a pressure is exerted on the finger artery. If the pressure is high enough, it will compress the artery and the amount of light registered by the sensor will increase. The amount of pressure necessary in the inflatable bladder to compress the artery is dependent on the blood pressure. By controlling the pressure of the inflatable bladder such that the diameter of the finger artery is kept constant, the blood pressure may be monitored in very precise detail as the pressure in the inflatable bladder is directly linked to the blood pressure. In a typical present day finger cuff implementation, a volume clamp system is used with the finger cuff. The volume clamp system typically includes a pressure generating system and a regulating system that includes: a pump, a valve, and a pressure sensor in a closed loop feedback system that are used in the measurement of the arterial volume. To accurately measure blood pressure, the feedback loop provides sufficient pressure generating and releasing capabilities to match the pressure oscillations of the patient's blood pressure.

Today, finger cuffs typically use an air bladder to apply pressure on the whole circumference of the finger. When the finger cuff is applied on the patient's finger, and the air bladder is inflated during use for blood pressure measurement, the finger is pressurized around its whole circumference.

It may be beneficial to utilize a bladder in a finger cuff that uses one or more partial bladders to implement different functions.

### SUMMARY

Embodiments of the invention may relate to a finger cuff that is applied on a patient's finger to be used in measuring the patient's blood pressure by a blood pressure measurement system utilizing the volume clamp method. The finger cuff may include a light emitting diode (LED) and photodiode to form an LED-PD pair to perform measurements of a plethysmogram signal to aid in measuring the patient's blood pressure; and a bladder. The bladder includes a bottom portion that abuts only the bottom portion of the patient's finger to apply concentrated pressure only on the bottom of the patient's finger near both of the patient's two arteries, wherein, when the finger cuff is placed around the patient's finger, the bottom portion bladder and LED-PD pair aid in measuring the patient's blood pressure by the blood pressure measurement system.

### BRIEF DESCRIPTION OF THE DRAWINGS

FIG. 1 is a diagram of an example of a blood pressure measurement system according to one embodiment.

FIG. 2 is a diagram illustrating an example of a finger cuff according to one embodiment.

FIGS. 3A-3B are diagrams illustrating cross-sectional views of the finger cuff with differing bladders attached to a patient's finger according to one embodiment.

FIG. 4 is a block diagram illustrating an example environment in which embodiments of the invention may be practiced.

### DETAILED DESCRIPTION

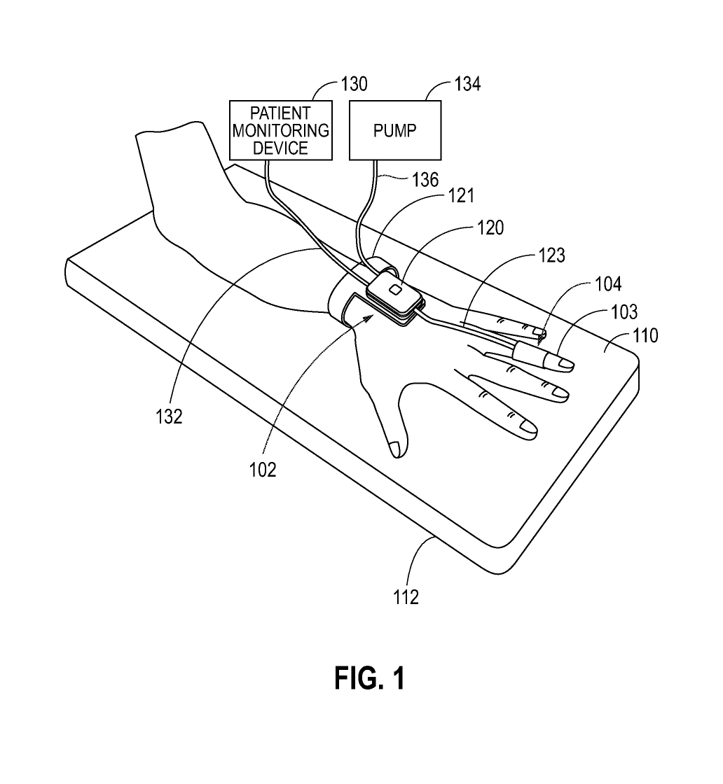

With reference to FIG. 1, which illustrates an example of a blood pressure measurement system according to one embodiment, a blood pressure measurement system 102 that includes a finger cuff 104 that may be applied to a patient's finger 103 and a blood pressure measurement controller 120, which may be attached to the patient's body (e.g., a patient's wrist or hand) is shown.

The blood pressure measurement system 102 may further be connected to a patient monitoring device 130, and, in some embodiments, a pump 134. Further, finger cuff 104 may include a bladder (not shown) and an LED-PD pair (not shown), which are conventional for finger cuffs.

In one embodiment, the blood pressure measurement system 102 may include a pressure measurement controller 120 that includes: a small internal pump, a small internal valve, a pressure sensor, and control circuitry. In this embodiment, the control circuitry may be configured to: control the pneumatic pressure applied by the internal pump to the bladder of the finger cuff 104 to replicate the patient's blood pressure based upon measuring the plethysmogram signal received from the LED-PD pair of the finger cuff 104. Further, the control circuitry may be configured to: control the opening of the internal valve to release pneumatic pressure from the bladder; or the internal valve may simply be an orifice that is not controlled. Additionally, the control circuitry may be configured to: measure the patient's blood pressure by monitoring the pressure of the bladder based upon the input from a pressure sensor, which should be the same as patient's blood pressure, and may display the patient's blood pressure on the patient monitoring device 130.

In another embodiment, a conventional pressure generating and regulating system may be utilized, in which, a pump 134 is located remotely from the body of the patient. In this embodiment, the blood pressure measurement controller 120 receives pneumatic pressure from remote pump 134 through tube 136 and passes on the pneumatic pressure through tube 123 to the bladder of finger cuff 104. Blood pressure measurement device controller 120 may also control the pneumatic pressure (e.g., utilizing a controllable valve) applied to the finger cuff 104 as well as other functions. In this example, the pneumatic pressure applied by the pump 134 to the bladder of finger cuff 104 to replicate the patient's blood pressure based upon measuring the plethysmogram signal received from the LED-PD pair of the finger cuff 104 (e.g., to keep the plethysmogram signal constant) and measuring the patient's blood pressure by monitoring the pressure of the bladder may be controlled by the blood pressure measurement controller 120 and/or a remote computing device and/or the pump 134 and/or the patient monitoring device 130 to implement the volume clamping method. In some embodiments, a blood pressure measurement controller 120 is not used at all and there is simply a connection from tube 136 from a remote pump 134 including a remote pressure regulatory system to finger cuff 104, and all processing for the pressure generating and regulatory system, data processing, and display is performed by a remote computing device.

Continuing with this example, as shown in FIG. 1, a patient's hand may be placed on the face 110 of an arm rest 112 for measuring a patient's blood pressure with the blood pressure measurement system 102. The blood pressure measurement controller 120 of the blood pressure measurement system 102 may be coupled to a bladder of the finger cuff 104 in order to provide pneumatic pressure to the bladder for use in blood pressure measurement. Blood pressure measurement controller 120 may be coupled to the patient monitoring device 130 through a power/data cable 132. Also, in one embodiment, as previously described, in a remote implementation, blood pressure measurement controller 120 may be coupled to a remote pump 134 through tube 136 to receive pneumatic pressure for the bladder of the finger cuff 104. The patient monitoring device 130 may be any type of medical electronic device that may read, collect, process, display, etc., physiological readings/data of a patient including blood pressure, as well as any other suitable physiological patient readings. Accordingly, power/data cable 132 may transmit data to and from patient monitoring device 130 and also may provide power from the patient monitoring device 130 to the blood pressure measurement controller 120 and finger cuff 104.

As can be seen in FIG. 1, in one example, the finger cuff 104 may be applied to a patient's finger 103 and the blood pressure measurement controller 120 may be attached on the patient's hand or wrist with an attachment bracelet 121 that wraps around the patient's wrist or hand. The attachment bracelet 121 may be metal, plastic, Velcro, etc. It should be appreciated that this is just one example of attaching a blood pressure measurement controller 120 and that any suitable way of attaching a blood pressure measurement controller to a patient's body or in close proximity to a patient's body may be utilized and that, in some embodiments, a blood pressure measurement controller 120 may not be used at all. It should further be appreciated that the finger cuff 104 may be connected to a blood pressure measurement controller described herein, or a pressure generating and regulating system of any other kind, such as a conventional pressure generating and regulating system that is located remotely from the body of the patient (e.g., a pump 134 located remotely from a patient). Any kind of pressure generating and regulating system can be used, including but not limited to the blood pressure measurement controller, and may be described simply as a pressure generating and regulating system that may be used with a finger cuff 104 including an LED-PD pair and a bladder to implement the volume clamping method.

Embodiments of the invention relate to applying concentrated pressure on the bottom of the patient's finger near the patient's arteries, instead of applying pressure all around the finger, as is the current practice. As will be described, this may be achieved by utilizing a bladder in a finger cuff that uses one or more partial bladders to implement different functions.

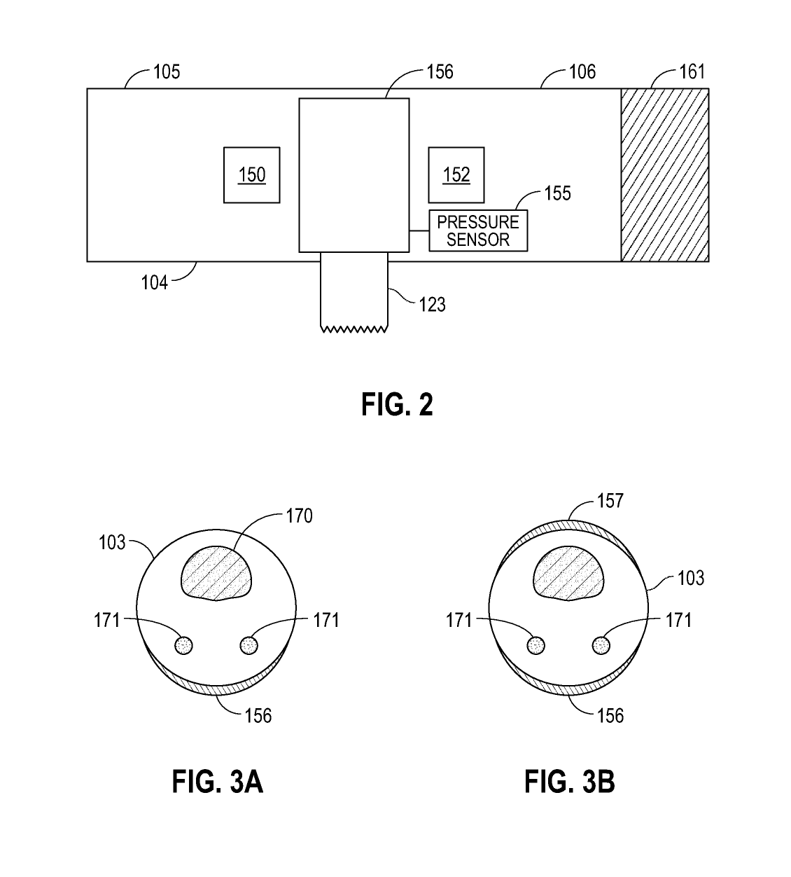

With additional reference to FIG. 2, in conjunction with the previously described FIG. 1, a finger cuff 104 that is connectable to a patient's finger 103, may be used in measuring the patient's blood pressure by a blood pressure measurement system 102 utilizing the volume clamp method. For example, the previously described blood pressure measurement system 102 may be utilized, or any suitable type of blood pressure measurement system.

An example finger cuff 104, as shown in FIG. 2, will now be described. Finger cuff 104 may include a first side 105 and a second side 106. In one example, for attachment purposes to a patient's finger 103, the second side 106 of the interior may have a first Velcro type portion 161 that connects with a second Velcro type portion on the exterior of the first side 105 of the finger cuff 104. It should be appreciated that this is just one example of an attachment mechanism and that any suitable type may be utilized (e.g., adhesive, tape, etc.). It should also be appreciated that any type of wrappable finger cuff, fixed type of finger cuff, or any type of finger cuff may be utilized, and this is just one example.

Further, finger cuff 104 may have a bladder 156 and an LED-PD pair 150 and 152. The LED-PD pair 150 and 152 may be used to perform measurements of a plethysmogram signal to aid in measuring the patient's blood pressure. Also, tube 123 may be connected to bladder 156 to apply pneumatic pressure to bladder 156. Again, it should be appreciated that this just one example of a finger cuff 104 and that any suitable finger cuff may be utilized with embodiments of the invention.

In particular, as shown in FIG. 2, bladder 156 may include a bottom portion that is denoted bottom portion bladder 156 that is sized to abut only the bottom portion of the patient's finger 103 to apply concentrated pressure only on the bottom of the patient's finger 103 near both of the patient's two arteries, wherein, when the finger cuff 104 is placed around the patient's finger 103, the bottom portion bladder 156 and the LED-PD pair 150 and 152 aid in measuring the patient's blood pressure by the blood pressure measurement system using the volume clamp method. As an example, as part of the volume clamp method, pneumatic pressure is applied to the bottom portion bladder 156 of the finger cuff 104 based upon measuring the plethysmogram signal received from the LED-PD pair 150 and 152 of the finger cuff 104 (e.g., to keep the plethysmogram signal constant) so that the pressure applied to the bottom portion bladder 156 and measured by the pressure sensor 155 should be correlated to the patient's blood pressure. It should be appreciated that the pressure sensor 155 can be connected to the bladder or tube at various locations. In particular, applying concentrated pressure on the bottom of the patient's finger near the arteries, by the bottom portion bladder 156, instead of applying pressure all around the finger, as is the current practice, provides many benefits, as will be described. Thus, in this configuration, a partial bottom portion bladder 156 is utilized. Further, in some embodiments, additional partial bladders may also be utilized for additional functions, as will be described.

With additional reference to FIGS. 3A and 3B, showing cross sections, a more particular description of embodiments of the invention will be described. With particular additional reference to FIG. 3A, the bottom portion bladder 156 abuts only the bottom portion of the patient's finger 105 to apply concentrated pressure only to the bottom of the patient's finger 103 near both of the patient's two arteries 171 and 171. The patient's bone 170 is also shown. Therefore, when the finger cuff 104 is placed around the patient's finger 103, the bottom portion bladder 156 and the LED-PD pair 150 and 152 may be used to aid in measuring the patient's blood pressure by the blood pressure measuring system and the volume clamp method, as has been described.

Therefore, in this implementation, the bottom portion bladder 156 forms only one pressure area where pressure is applied directly under both of the patient's two fingers arteries 171 and 171. Also, as has been described, the pressure sensor 155 may be coupled to the bottom portion bladder 156 to measure the air pressure of the bottom portion bladder 156 to aid in determining the patient's blood pressure as part of the volume clamp method by the blood pressure measurement system. As an example, as part of the volume clamp method, pneumatic pressure is applied to the bottom portion bladder 156 of the finger cuff 104 based upon measuring the plethysmogram signal received from the LED-PD pair 150 and 152 of the finger cuff 104 (e.g., to keep the plethysmogram signal constant) so that the pressure applied to the bottom portion bladder 156 and measured by the pressure sensor 155 should be correlated to the patient's blood pressure.

As can be particularly seen, in FIG. 3A, the bladder including a particular bottom portion bladder 156 applies concentrated pressure only on the bottom of the patient's finger 103 near the patient's two arteries 171 and 171. In this way, only one pressure area is applied directly under patient's two arteries 171 and 171. Therefore, this singular bottom portion bladder 156 can be used to apply pressure and for measurement purposes with the pressure sensor for use with the volume clamp method by the blood pressure measurement system.

With additional reference to FIG. 3B, a bladder configuration/implementation may further include a second top portion bladder 157 for use in conjunction with the first bottom portion bladder 156 such that two different pressure areas on the patient's finger 103 may be utilized. Thus, the second top portion bladder 157 may be on the opposite side of the first bottom portion bladder 156 and is on the top side of the patient's finger 103. As will be described, the second top portion bladder 157 may be used to compensate for variations in finger shape, finger size, and volume change due to pressure measurement through time.

Therefore, in one embodiment, the bottom portion bladder 156 may be used to apply pressure to the patient's arteries 171 and 171 of the patient's finger 103 for use in blood pressure measurement via the volume clamp method by the blood measurement system, as previously described. On the other hand, the second pressure area provided by the top portion bladder 157 on top of the patient's finger 103 on the opposite side of the first pressure area provided by the bottom portion bladder 156 for blood pressure measurement provides discrete and different features than the bottom portion bladder 156 (i.e., unrelated to blood pressure measurement). In particular, the second pressure area provided by the top portion bladder 157 can be used to compensate for variations in the patient's finger shape, finger size, and volume change (e.g., due to pressure measurement through time). Further, the top portion bladder 157 may be used to increase the comfort of the use of the finger cuff to the patient. Moreover, the second pressure area provided by the top portion bladder 157 may be used to stabilize the blood pressure measurement of the bottom portion bladder 156. For example, the top portion bladder 157 may be set to a constant pressure or a variable pressure that has been determined to optimize the accuracy of the blood pressure measurement in the volume clamp implementation that is reliant upon the performance of the bottom portion bladder 156.

Many benefits may be provided by the use of the bottom portion bladder 156 alone, or in conjunction with, the opposite top portion bladder 157. These benefits may include: improved measurement accuracy due to the concentrated pressure application of the bottom portion bladder 156; and simplified product design for the finger cuff. For example, the bottom portion bladder 156 itself is smaller and therefore less costly than current bladders for finger cuffs. Further, the accompanying pressure sensor 155 and other components of the finger cuff may also have a reduced size. Moreover, with this type of implementation, a one size fits all finger cuff may be implemented. In other words, separately sized finger cuffs (e.g., small, medium, large, etc.) for differently sized fingers may no longer be required because the finger cuff according to embodiments of the invention may be fitted to most any sized finger. In particular, the top portion bladder 157 may be adjusted in size to provide fitting to most any sized finger, such that the finger cuff may be one size fits all. Other benefits such as, ease of use, versatility (e.g., enabling other opportunities for product design), reduced product costs, increased comfort for the patient, are also provided.

Further, it should be appreciated that the pressure applied on the pressure areas by the bottom portion bladder 156 and the top portion bladder 157 may be generated by all types of medium—such as, air, liquid, electricity, etc. The previously described implementations can work with any of these types of mediums. Also, these types of pressure application areas utilizing differently sized bladder portions may be utilized with other extremity areas of the human body—such as, wrists, arms, ankles, legs, etc.

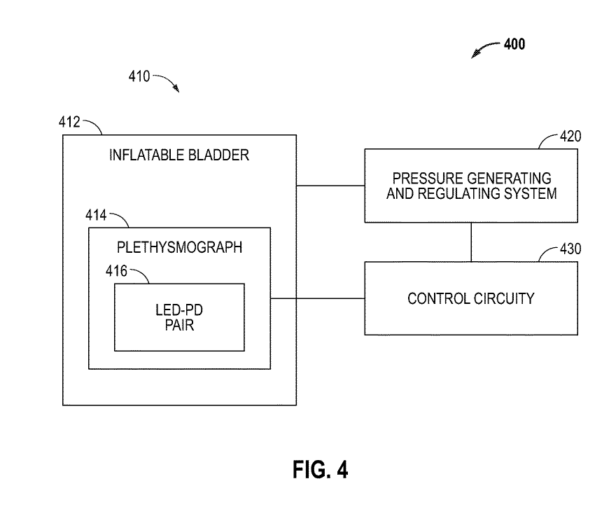

FIG. 4 is a block diagram illustrating an example environment 400 in which embodiments of the invention may be practiced. As shown, finger cuff assembly 410 may include an inflatable bladder 412 and a plethysmograph 414. The inflatable bladder 412 may be pneumatically connected to a pressure generating and regulating system 420. The pressure generating and regulating system 420 may generate, measure, and regulate pneumatic pressure that inflates or deflates the inflatable bladder (e.g., the bottom portion bladder 156), and may include elements such as a pump, a valve, a sensor, control circuitry, and/or other suitable elements. When the bottom portion bladder 156 is inflated, a pressure is applied to the patient's finger. In particular, the volume clamp method is applied, in which, pneumatic pressure is applied to the bottom portion bladder 156 of the finger cuff based upon measuring the plethysmograph signal received from the LED-PD pair 416 (e.g., to keep the plethysmograph signal constant) so that the pressure applied to the bottom portion bladder 156 and measured by the pressure sensor should be correlated to the patient's blood pressure. The plethysmograph 414 may make continuous volumetric measurements (or plethysmogram) of arterial blood flows within the finger and, in one embodiment, may include the LED-PD pair 416. The LED may be used to illuminate the finger skin and light absorption or reflection may be detected with the photodiode. Therefore, the plethysmogram may be generated based on the signal received from the photodiode. The pressure generating and regulating system 420 and the plethysmograph 414 may be connected to a control circuitry 430. The control circuitry 430 may instruct the pressure generating and regulating system 420 to inflate or deflate the bottom portion bladder 156 and may receive data from the plethysmograph 414 and may carry out necessary data manipulations. Also, in some embodiments, the control circuitry 430 may instruct the pressure generating and regulating system 420 to inflate or deflate the top portion bladder 157.

It should be appreciated that aspects of the invention previously described may be implemented in conjunction with the execution of instructions by processors, circuitry, controllers, control circuitry, etc. As an example, control circuitry may operate under the control of a program, algorithm, routine, or the execution of instructions to execute methods or processes in accordance with embodiments of the invention previously described. For example, such a program may be implemented in firmware or software (e.g. stored in memory and/or other locations) and may be implemented by processors, control circuitry, and/or other circuitry, these terms being utilized interchangeably. Further, it should be appreciated that the terms processor, microprocessor, circuitry, control circuitry, circuit board, controller, microcontroller, etc., refer to any type of logic or circuitry capable of executing logic, commands, instructions, software, firmware, functionality, etc., which may be utilized to execute embodiments of the invention.

The various illustrative logical blocks, processors, modules, and circuitry described in connection with the embodiments disclosed herein may be implemented or performed with a general purpose processor, a specialized processor, circuitry, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, circuitry, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module/firmware executed by a processor, or any combination thereof. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A finger cuff attachable to a patient's finger to be used in measuring the patient's blood pressure by a blood pressure measurement system utilizing the volume clamp method, the finger cuff comprising:
a light emitting diode (LED) and photodiode to form an LED-PD pair to perform measurements of a plethysmogram signal to aid in measuring the patient's blood pressure; and
a bladder having a bottom portion that abuts only the bottom portion of the patient's finger to apply concentrated pressure only on the bottom of the patient's finger near both of the patient's two arteries, wherein, when the finger cuff is placed around the patient's finger, the bottom portion bladder and LED-PD pair aid in measuring the patient's blood pressure by the blood pressure measurement system.

2. The finger cuff of claim 1, wherein the bottom portion bladder forms only one pressure area where pressure is applied directly under both of the patient's finger arteries.

3. The finger cuff of claim 2, wherein a pressure sensor coupled to the bottom portion bladder measures the pressure of the bottom portion bladder to aid in determining the patient's blood pressure as part of the volume clamp method by the blood pressure measurement system.

4. The finger cuff of claim 3, further comprising a second top portion bladder on the opposite side of the bottom portion bladder on the top portion of the patient's finger.

5. The finger cuff of claim 4, wherein the second top portion bladder is used to compensate for variations in finger shape, finger size, and volume change due to pressure measurement through time.

6. The finger cuff of claim 5, wherein the second top portion bladder increases the comfortability to the patient.

7. The finger cuff of claim 4, wherein the second top portion functions to stabilize the blood pressure measurement of the bottom portion bladder.

8. A method to measure a patient's blood pressure by a blood pressure measurement system utilizing a finger cuff and the volume clamp method, the finger cuff comprising a light emitting diode (LED) and photodiode to form an LED-PD pair to perform measurements of a plethysmogram signal to aid in measuring the patient's blood pressure and a bladder having a bottom portion, the method comprising:
inserting the patient's finger into the finger cuff such that the LED-PD pair aids in measuring the patient's blood pressure by the blood pressure measurement system; and
inserting the patient's finger such that the bottom bladder portion abuts against only the bottom portion of the patient's finger and the bottom bladder portion applies concentrated pressure only on the bottom of the patient's finger near both of the patient's two arteries, such that, the bottom portion bladder and LED-PD pair aid in measuring the patient's blood pressure by the blood pressure measurement system.

9. The method of claim 8, wherein the bottom portion bladder forms only one pressure area where pressure is applied directly under both of the patient's finger arteries.

10. The method of claim 9, wherein a pressure sensor coupled to the bottom portion bladder measures the pressure of the bottom portion bladder to aid in determining the patient's blood pressure as part of the volume clamp method by the blood pressure measurement system.

11. The method of claim 10, further comprising a second top portion bladder on the opposite side of the bottom portion bladder on the top portion of the patient's finger.

12. The method of claim 11, wherein the second top portion bladder is used to compensate for variations in finger shape, finger size, and volume change due to pressure measurement through time.

13. The method of claim 12, wherein the second top portion bladder increases the comfortability to the patient.

14. The method of claim 11, wherein the second top portion functions to stabilize the blood pressure measurement of the bottom portion bladder.

15. A blood pressure measurement system utilizing the volume clamp method, the blood pressure measurement system comprising:
a finger cuff connectable to a patient's finger to be used in measuring the patient's blood pressure by the blood pressure measurement system utilizing the volume clamp method, the finger cuff comprising:
a light emitting diode (LED) and photodiode to form an LED-PD pair to perform measurements of a plethysmogram signal to aid in measuring the patient's blood pressure; and
a bladder having a bottom portion that abuts only the bottom portion of the patient's finger to apply concentrated pressure only on the bottom of the patient's finger near both of the patient's two arteries, wherein, when the finger cuff is placed around the patient's finger, the bottom portion bladder and LED-PD pair aid in measuring the patient's blood pressure by the blood pressure measurement system.

16. The blood pressure measurement system of claim 15, wherein the bottom portion bladder forms only one pressure area where pressure is applied directly under both of the patient's finger arteries.

17. The blood pressure measurement system of claim 16, wherein a pressure sensor coupled to the bottom portion bladder measures the pressure of the bottom portion bladder to aid in determining the patient's blood pressure as part of the volume clamp method by the blood pressure measurement system.

18. The blood pressure measurement system of claim 17, further comprising a second top portion bladder on the opposite side of the bottom portion bladder on the top portion of the patient's finger.

19. The blood pressure measurement system of claim 18, wherein the second top portion bladder is used to compensate for variations in finger shape, finger size, and volume change due to pressure measurement through time.

20. The blood pressure measurement system of claim 19, wherein the second top portion bladder increases the comfortability to the patient.

21. The blood pressure measurement system of claim 18, wherein the second top portion functions to stabilize the blood pressure measurement of the bottom portion bladder.
